(19) United States
(12) Patent Application Publication
(10) Pub. No.: US 2025/0316361 A1
(43) Pub. Date: Oct. 9, 2025

(54) SYSTEMS AND METHODS FOR ESTIMATING, FROM FOOD FREQUENCY QUESTIONNAIRE BASED NUTRIENTS INTAKE DATA, THE RELATIVE AMOUNTS OF FAECALIBACTERIUM PRAUSNITZII (FPRAU) IN THE GUT MICROBIOME ECOSYSTEM AND ASSOCIATED RECOMMENDATIONS TO IMPROVE FAECALIBACTERIUM PRAUSNITZII

(71) Applicant: SOCIETE DES PRODUITS NESTLE S.A., Vevey (CH)

(72) Inventor: SHAILLAY KUMAR DOGRA, Epalinges (CH)

(21) Appl. No.: 18/558,955

(22) PCT Filed: May 4, 2022
(86) PCT No.: PCT/EP2022/061952

§ 371 Date: Nov. 3, 2023

(30) Foreign Application Priority Data
May 6, 2021 (EP) 21172422.4

(51) Int. Cl. G16H 20/60; G16H 10/20; G16H 50/20

(52) U.S. Cl. CPC G16H 20/60; G16H 10/20; G16H 50/20

(57) ABSTRACT

The present invention relates to systems and methods for estimating an individual's Faecalibacterium prausnitzii (Fprau) amounts and for providing personalized recommendations to maintain or improve the Fprau. In several embodiments of the invention, the individual's Fprau amounts are estimated based on their Food FrequencyQuestionnaire (FFQ) records. In several embodiments, the methods are implemented by a computer system. In several embodiments of the invention, personalized recommendations and dietary advice are given to the individual to maintain or improve said individual's Fprau.

### CROSS REFERENCE TO RELATED APPLICATIONS

The present application is a National Stage of International Application No. PCT/EP2022/061952, filed on May 4, 2022, which claims priority to European Patent Application No. 21172422.4, filed on May 6, 2021, the entire contents of which are being incorporated herein by reference.

### FIELD OF THE INVENTION

The present invention relates to systems and methods for estimating an individual's Faecalibacterium prausnitzii (Fprau) amounts. In several embodiments of the invention, the individual's Fprau amounts are estimated based on nutrients data derived from the individual's Food Frequency Questionnaire (FFQ) records. In several embodiments, the methods are implemented by a computer system. In several embodiments of the invention, personalized recommendations and dietary and nutrition advice are given to the individual to maintain or improve said individual's Fprau amounts.

### BACKGROUND TO THE INVENTION

Faecalibacterium prausnitzii (Fprau) is an important bacteria in the human gut microbiome ecosystem with an associative to causative role in different conditions such as its importance in human health (Miguel, S et al. Current opinion in microbiology, 2013; Ferreira-Halder, C V et al. Clinical gastroenterology, 2017); anti-inflammatory (Quevrain, E et al. Gut, 2016; Sokol, H et al. PNAS, 2008); ulcerative colitis (Machiels, K et al. Gut, 2014); crohn's disease (Takahashi, K et al. Digestion. 2016); children allergy such as asthma (Demirci, M et al. Allergologia et immunopathologia, 2019); IBD (Zhao H, Xu H, Chen S, He J, Zhou Y, Nie Y., 2020. J Gastroenterol Hepatol.; Machiels K et al., Gut. 2014); frailty (Jackson M A et al. Genome Med. 2016) and so on.

Additionally, Fprau is affected in multiple stressful conditions to the gut microbiome ecosystem such as a drastic diet change or antibiotics usage. For example, Mardinoglu et al. Cell Metabolism 2018 showed a decrease in Fprau under a ketogenic diet challenge. Similarly, Palleja et al. Nature Microbiology 2018 showed a decrease in Faecalibacterium prausnitzii (Fprau) under an antibiotic challenge. Furthermore, David et al. Nature 2014, provided evidence of a decrease in Fprau abundance under high fat diet challenge.

Typically, assessment of the bacteria in the gut microbiome ecosystem requires collection of fecal samples, storage and processing of samples, laboratory steps such as DNA extraction and sequencing, complex bioinformatics analyses and scientific assessment. This costs money, time, effort and needs specialized skills and expertise not necessarily available everywhere, easily accessible to everyone. Additionally, many adults are reluctant to provide their fecal sample.

There is thus a need for non-invasive and simpler way to estimate relative amounts of Faecalibacterium prausnitzii (Fprau), and methods to promote Fprau in human gut microbiome ecosystem.

### SUMMARY OF THE INVENTION

The present inventors have found a simpler way to estimate relative amounts of Faecalibacterium prausnitzii (Fprau) from nutrients intake data. The key steps of the invention here are: (i) an individual's responses to certain food questions; (ii) estimation of nutrient intake amounts for the said individual; (iii) use of machine-learning based models; (iv) prediction of estimated relative amounts of Faecalibacterium prausnitzii (Fprau).

Accordingly, the present invention generally relates to a method for determining the gut Faecalibacterium prausnitzii (Fprau) status comprising:
(i) assessing the relative amounts of Fprau in an individual's gut microbiome ecosystem; and (ii) accordingly providing recommendations to maintain or improve Fprau relative amounts.

In another aspect, the present invention relates to a method for optimizing one or more dietary interventions for a subject comprising:
(i) determining the Fprau status of a subject according to a method as defined in any one of claims 1 to 5; and
(ii) applying the dietary intervention to the subject.

The methods and systems of the present invention advantageously implement Artificial Intelligence based Machine Learning methods to estimate an individual's gut microbiome Fprau amounts from nutrients data derived from Food Frequency Questionnaires (FFQ).

One advantage of the present invention is that the individual does not need to provide a biological sample to get an estimate of their Fprau amounts. Instead, this is done by using predictive models based on the data provided by the user in terms of responses to a set of food frequency questionnaires in order to discern nutrients intake as predictive features.

In another embodiment, the present invention relates to a kit comprising a food frequency questionnaire to determine the nutrients intake to predict the Fprau status of said subject and a computer-implemented tool for dietary recommendation to maintain or improve Fprau relative amounts.

One advantage of several embodiments of the invention is that for the Fprau status assessment, individual user's questionnaire responses are evaluated to personalize the recommendations and advice to maintain or improve the individual's Fprau status.

Various embodiments of the disclosed system display a dashboard or other appropriate user interface to a user that is customized based on the user's inputs to the questionnaire, estimated Fprau amounts, and personalized advise to maintain or improve the Fprau.

In some embodiments, the disclosed system may be linked to automatically collect the required input data from dietary records captured by the user in various formats such as food diary or apps that log eating records.

In some embodiments, the systems and methods disclosed herein can be also used by nutritionists, health-care professionals, beyond the individual users.

Further advantages of the instant disclosure will be apparent from the following detailed description and associated figures.

### DESCRIPTION OF FIGURES

FIG. 1—ROC performance of a Low versus notLow model (I)

The ROC performance of a Low versus notLow model for Fprau amounts with the definition of the bins based on (mean−1*std) vs rest. ROC for (A) Train in cross-validation mode (B) holdout/Test set.

FIG. 2—ROC performance of a Low versus notLow model (II)

The ROC performance of a Low versus notLow model for Fprau amounts with the definition of the bins based on first/lowest quartile vs rest. ROC for (A) Train in cross-validation mode (B) holdout/Test set.

FIG. 3—Features important for the Low versus notLow model (I)

The important features and their association with Fprau are shown in A and B, respectively.

FIG. 4—Features important for the Low versus notLow model (II)

The important features and their association with Fprau are shown in A and B, respectively

FIG. 5—SHAP dependence plots for key features in a Low versus notLow model

The SHAP dependence plots, for a Low versus notLow model on Fprau amounts with the definition of the bins based on quartiles, are shown for key example features. The reference class here was “Low”, so the positive coefficients of SHAP value for the corresponding x-values of the feature indicate how much the model was affected by this feature in predicting the “Low” class.

FIG. 6—Results of F. prau quantity determined by a quantitative PCR technique.

A) samples collected after 24 hours and B) after 48 hours.

FIG. 7—F. prau ASV6 responded to inulin, PuMP_full and vit Bs+inositol. A) samples collected after 24 hours and B) after 48 hours.

### DETAILED DESCRIPTION OF THE INVENTION

### Definitions

Some definitions are provided hereafter. Nevertheless, definitions may be located in the “Embodiments” section below, and the above header “Definitions” does not mean that such disclosures in the “Embodiments” section are not definitions.

All percentages expressed herein are by weight of the total weight of the composition unless expressed otherwise. As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number. All numerical ranges herein should be understood to include all integers, whole or fractions, within the range. Moreover, these numerical ranges should be construed as providing support for a claim directed to any number or subset of numbers in that range.

The words “comprise,” “comprises” and “comprising” are to be interpreted inclusively rather than exclusively. Likewise, the terms “include,” “including” and “or” should all be construed to be inclusive, unless such a construction is clearly prohibited from the context. Nevertheless, the compositions disclosed herein may lack any element that is not specifically disclosed herein. Thus, a disclosure of an embodiment using the term “comprising” includes a disclosure of embodiments “consisting essentially of” and “consisting of” the components identified.

The terms “at least one of” and “and/or” used in the respective context of “at least one of X or Y” and “X and/or Y” should be interpreted as “X,” or “Y,” or “X and Y.” For example, “at least one of inositol or sorbitol” and “inositol and/or sorbitol” should be interpreted as “inositol without sorbitol,” or “sorbitol without inositol,” or “inositol without sorbitol.”

Where used herein, the terms “example” and “such as,” particularly when followed by a listing of terms, are merely exemplary and illustrative and should not be deemed to be exclusive or comprehensive. As used herein, a condition “associated with” or “linked with” another condition means the conditions occur concurrently, preferably means that the conditions are caused by the same underlying condition, and most preferably means that one of the identified conditions is caused by the other identified condition.

The relative terms “promote,” “improve,” “increase,” “enhance” and the like refer to an enhanced status of F. prausnitzii in the microbiome of the subject, after administration of a the composition disclosed herein (which comprises sorbitol and/or inositol), relative to the status of F. prausnitzii in the microbiome of the subject obtained by administration of a recommendation according to the present invention. This enhanced status of F. prausnitzii in the microbiome of the subject can be characterized by at least one or more of (i) a higher total amount of F. prausnitzii in the microbiome of the subject (i.e., total cfu of Faecalibacterium prausnitzii) or (ii) a higher relative percentage of Faecalibacterium prausnitzii compared to the other bacteria in the microbiome of the subject (i.e., cfu of Faecalibacterium prausnitzii/cfu of other bacteria).

As used herein, the terms “food,” “food product” and “food composition” mean a product or composition that is intended for oral ingestion by a human or other mammal and comprises at least one nutrient for the human or other mammal.

“Nutritional compositions” and “nutritional products,” as used herein, include any number of food ingredients and possibly optional additional ingredients based on a functional need in the product and in full compliance with all applicable regulations. The optional ingredients may include, but are not limited to, conventional food additives, for example one or more, acidulants, additional thickeners, buffers or agents for pH adjustment, chelating agents, colorants, emulsifies, excipient, flavor agent, mineral, osmotic agents, a pharmaceutically acceptable carrier, preservatives, stabilizers, sugar, sweeteners, texturizers, and/or vitamins. The optional ingredients can be added in any suitable amount.

As used herein, “lifestyle characteristic” is meant any lifestyle choice made by a subject, this includes all dietary intake data, activity measures or data from questionnaires of lifestyle, motivation or preferences. In one embodiment, the lifestyle characteristic is whether the subject is a alcohol drinker or a non-drinker. In another embodiment, the lifestyle characteristic is whether the subject is a vegetarian or omnivore.

In some embodiments, the term “nutrient” as used herein refers to compounds having a beneficial effect on the body e.g. to provide energy, growth or health. The term includes organic and inorganic compounds. As used herein the term nutrient may include, for example, macronutrients, micronutrients, essential nutrients, conditionally essential nutrients and phytonutrients. These terms are not necessarily mutually exclusive. For example, certain nutrients may be defined as either a macronutrient or a micronutrient depending on the particular classification system or list. The expression “at least one nutrient” or “one or more nutrients” means, for example, one, two, three, four, five, ten, 20 or more nutrients.

In various embodiments, the term “macronutrient” is used herein consistent with its well understood usage in the art, which generally encompasses nutrients required in large amounts for the normal growth and development of an organism. Macronutrients in these embodiments may include, but are not limited to, carbohydrates, fats, proteins, amino acids and water. Certain minerals may also be classified as macronutrients, such as calcium, chloride, sodium, or potassium.

In various embodiments, the term “micronutrient” is used herein consistent with its well understood usage in the art, which generally encompasses compounds having a beneficial effect on the body, e.g. to provide energy, growth or health, but which are required in only minor or trace amounts. The term in such embodiments may include or encompass both organic and inorganic compounds, e.g. individual amino acids, nucleotides and fatty acids; vitamins, antioxidants, minerals, trace elements, e.g. iodine, and electrolytes, e.g. sodium chloride, and salts thereof.

In various embodiments, the term “essential nutrient” is used herein consistent with its well understood usage in the art. Essential nutrients are unable to be synthesized internally either at all, or in sufficient quantities, and so must be consumed by an organism from its environment. These include essential fatty acids, essential amino acids, vitamins, and certain dietary minerals. For example, there are two essential fatty acids for humans: alpha-linolenic acid (an omega-3 fatty acid) and linoleic acid (an omega-6 fatty acid). There are nine out of the twenty amino acids that cannot be endogenously synthesized by humans: phenylalanine, valine, threonine, tryptophan, methionine, leucine, isoleucine, lysine, and histidine and these are considered essential amino acids.

In various embodiments, the term “conditionally essential nutrient” is used herein consistent with its well understood usage in the art. Conditionally essential nutrients are certain organic molecules that can normally be synthesized by an organism, but under certain conditions such biosynthesis is not enough to prevent a deficiency syndrome. For example, choline, inositol, taurine, arginine, glutamine and nucleotides are classified as conditionally essential, particularly for neonatal diet and metabolism.

In various embodiments, the term “non-essential nutrient” is used herein consistent with its well understood usage in the art. Non-essential nutrients are those nutrients that can be made by the body; they may often also be absorbed from consumed food. Non-essential nutrients are substances within foods can still have a significant impact on health, whether beneficial or toxic. For example, most dietary fiber is not absorbed by the human digestive tract but is important in maintaining the bulk of a bowel movement to avoid constipation or has recently become evident to have a beneficial impact on the gut microbiome with various bacteria having differing capacities or preferences to utilize fibers.

In various embodiments, the term “deficiency” is used herein consistent with its well understood usage in the art. Deficiencies can be due to a number of causes including inadequacy in nutrient intake called dietary deficiency, or conditions that interfere with the utilization of a nutrient within an organism. Some of the conditions that can interfere with nutrient utilization include problems with nutrient absorption, substances that cause a greater than normal need for a nutrient, conditions that cause nutrient destruction, and conditions that cause greater nutrient excretion.

In various embodiments, the term “toxicity” is used herein consistent with its well understood usage in the art. Nutrient toxicity occurs when an excess of a nutrient does harm to an organism.

A “subject” or “individual” is a mammal, preferably a human, but it can also be a pet animal such as a dog or cat.

In some embodiments, Low, notLow Fprau bins are defined as “Low” as being below the first or lower quartile of the population Fprau distribution and “notLow” as the rest of the distribution.

In some embodiments, High, notHigh Fprau bins are defined as “High” being above the third or upper quartile of the population Fprau distribution and “notHigh” as the rest of the distribution.

In some embodiments, Low, High Fprau bins are defined as: “Low” being below the first or lower quartile on the Fprau distribution and “High” being above the third or upper quartile on Fprau distribution.

In some embodiments, Low Fprau bin is defined as the data which is less than the mean minus the standard deviation on the Fprau distribution and notLow Fprau bin as the rest of the data.

In some embodiments, High Fprau bin is defined as the data which is more than the mean plus the standard deviation on the Fprau distribution and notHigh Fprau bin as the rest of the data.

In some embodiments, Low, High Fprau bins are defined as: “Low” being defined as the data which is less than the mean minus the standard deviation on the Fprau distribution and “High” being defined as the data which is more than the mean plus the standard deviation on the Fprau distribution.

In some embodiments, Low, notLow, High, notHigh Fprau bins are defined as in different population data sets with different numerical cut-offs, based on the data distribution, that would be apparent to a person skilled in the art.

It should be appreciated that the groups can be defined in many other possible ways which are variations of the above but with somewhat different definitions such as median/mean+/−1 standard deviation or median/mean+/−½ standard deviation or median/mean+/−½ inter-quartile range or a different % of data points going into the bins than what has been mentioned above, which is apparent to a person skilled in the art of data analytics.

“Receiver Operating Characteristic” (ROC) curve is one of the best-developed statistical tools for describing the performance of diagnostic tests measured on continuous scale. ROC use is based on having two outcomes from the prediction. Numerical indices of the ROC curves were used to summarize the curves. These summary measures were also used for comparing the ROC curves.

“Area Under the ROC curve” (AUC) is the most widely used summary measure. A perfect prediction model with the ideal ROC curve has the value AUC=1.0, while random prediction model has AUC=0.5. ROC curve AUC value moving from 0.5 towards 1.0 indicates improving and better performance of prediction models.

Many other measures of model performance can be calculated on the confusion matrix, such as true positives (TP), false positives (FP), true negatives (TN), false negatives (FN), total predicted positive, total predicted negative, total actual positive, total actual negative, sensitivity/hit rate/recall/true positive rate (TPR), specificity/selectivity/true negative rate (TNR), prevalence, precision/positive predictive value (PPV), negative predictive value (NPV), miss rate/false negative rate (FNR), fall-out/false positive rate (FPR), false discovery rate (FDR), false omission rate (FOR), prevalence threshold (PT), threat score (TS)/critical success index (CSI), accuracy (ACC), balanced accuracy (BA), random accuracy, total accuracy, FI score, Matthews correlation coefficient (MCC), Fowlkes Mallows index (FM), Informedness/Bookmaker informedness (BM), Markedness (MK)/deltaP, Positive likelihood ratio (LR+), Negative likelihood ratio (LR−), Diagnostic odds ratio (DOR), and Kappa.

AUC-ROC is the area under the curve which is created by plotting the true positive rate against the false positive rate at various probabilities. AUC-PR is the area under the precision recall curve.

The term “feature” is used repeatedly herein. In some embodiments, the term “feature” as used herein refers to the input parameters to the models. The term includes responses obtained from the sets of questionnaires, for example, nutrients intake derived from Food Frequency Questionnaires. These features are not necessarily mutually exclusive.

In various embodiments, the user-specific (or population-specific) inputs to the disclosed system are programmable and configurable, and include gender, age, weight, height, physical activity level, whether obese, and the like.

### EMBODIMENTS

The inventors have shown that a predictive tool can be created that is based on features obtained from questionnaire, such as food frequency questionnaires converted to nutrients intake, and that allows to predict the gut Fprau status, for example, Low or notLow.

In a first embodiment, the present invention provides a method for determining the gut Faecalibacterium prausnitzii (Fprau) status comprising:
(i) determining the gut Fprau status in a subject and
(ii) providing recommendations to improve, or maintain Fprau status in said subject.

In one embodiment, the methods and systems of the present invention implement Artificial Intelligence based Machine Learning methods to estimate an individual's gut microbiome Fprau amounts from nutrients data derived from Food Frequency Questionnaires (FFQ).

In another embodiments, this is done by using predictive models based on the data provided by the user in terms of responses to a set of food frequency questionnaires in order to discern nutrients intake as predictive features.

In further embodiments, the determination of gut Fprau status may additionally be provided by a biological sample to quantify the microbiome diversity of said subject.

In preferred embodiments, the present invention determines the Fprau status of an individual in relation to their position within the distribution in a larger population. For example, in terms of either having it Low or notLow, High or notHigh or when combined together to determine Low, Medium, High and possibly cross-confirmed by another Low vs. High assessment, where Low, High or Low, notLow or High, notHigh are defined in various ways based on the distribution seen for a large-sized general population such as in the American Gut Project (AGP) (McDonald D, et al. mSystems. 2018).

Once the Faecalibacterium prausnitzii (Fprau) relative amounts have been determined, the systems and methods of the invention contribute to maintaining and improving the Fprau status, or enhancing the growth of Fprau, by providing recommendations such as nutritional supplements, diet recommendations, menu recommendations and recipe recommendations to improve or maintain the Fprau amounts in the gut ecosystem.

In a preferred embodiment, some of the interventions to maintain or improve its abundance and function are provided as example 5.

Additionally, other methods are known in the art. This may be:
(i) consuming dietary fiber (Lin D et al. Br J Nutr. 2018; Benus R F et al. Br J Nutr. 2010);
(ii) following mediterraen diet (Gutierrez-Diaz I et al. J Agric Food Chem. 2017; Meslier V et al. Gut. 2020; Hero C et al. J Clin Endocrinol Metab. 2016);
(iii) following other diets (Verhoog, S et al. Nutrients, 2019; Fritsch J et al. 2020; Kahleova H et al. Nutrients. 2020; Medina-Vera I et al. Diabetes Metab. 2019);
(iv) consuming food containing pectins such as fruits (Lopez-Siles, M et al. Applied and environmental microbiology, 2012); (v) drinking red wine (Moreno-Indias I et al. Food Funct. 2016); (vi) consuming raisins (Wijayabahu A T et al. Nutr J. 2019) and so on.

Additional interventions to benefit Fprau can be through vitamins or probiotics but currently human clinical trial data seems lacking in this regard.

In another embodiment, the present method involves evaluation of feature parameters related to gut Fprau status as low, medium or high.

In one embodiment of the invention, improvements or maintenance of Fprau amounts can be determined from a biological sample taken from the subject, before and after the recommendations of the present invention, by measurement of parameters of microbial species in the intestine, particularly for Fprau. Thus, it can be determined over time, the Fprau maintenance or improvements after the individual has followed, for example, the nutrients, diet, menu and recipe recommendations of the present invention.

In various embodiments, the system disclosed herein provides recommendations of supplements, food items, menus or recipes indicating the nutritional impact for the Fprau. In these embodiments, the system determines and stores one or more indications of the needs of the individual for whom the recommendations are being calculated, for an individual over a given period of time such as a meal, an entire day, a week or a month.

In further embodiments, individuals may provide their own weighting values tailored to their own personal choices and health conditions. With these personalized ranges and/or weighting values, the disclosed system can then calculate a completely personalized advice for maintaining or improving the individual's Fprau status.

In an embodiment, the disclosed system includes or is connected to a database containing food items, menus or recipes and respective nutrient content. In this embodiment, the disclosed system includes a fuzzy search feature that enables a user to enter a consumed (or to-be consumed) food, and thereafter searches the database to find a closest item to the user-provided item. The disclosed system, in this embodiment, uses stored nutritional information about the matched food item to determine whether it is a microbiome-friendly item particularly for Fprau.

In various embodiments, the disclosed system further includes an interface (e.g., a graphical user interface) to display the amount of each nutrient available in each food composing the diet and displays the amount of energy available to be consumed. In some embodiments, this interface enables users to modify the amount of various foods or energy to be consumed. In other embodiments, the system is configured to determine amounts of food or energy consumed using non-user-input data, such as by scanning one or more bar codes, QR codes, or RFID tags, image recognition systems, or by tracking items ordered from a menu or purchased at a grocery store.

Various embodiments of the disclosed system display a dashboard or other appropriate user interface to a user that is customized based on the user's needs. In embodiments of the system disclosed herein, a graphical user interface is provided which advantageously enables, for the first time, users to input data about his responses to the sets of questionnaires and for him to see an indication of a score, based appropriately on prediction, that reflects overall placement of his status in the generally seen distribution of Fprau amounts.

In some embodiments, the disclosed system may be linked to automatically collect the required input data from dietary records captured by the user in various formats such as food diary or apps that log eating records.

All the disclosed methods and procedures described in this disclosure can be implemented using one or more computer programs or components. These components may be provided as a series of computer instructions on any conventional computer readable medium or machine-readable medium, including volatile and non-volatile memory, such as RAM, ROM, flash memory, magnetic or optical disks, optical memory, or other storage media. The instructions may be provided as software or firmware and may be implemented in whole or in part in hardware components such as ASICs, FPGAS, DSPs, or any other similar devices. The instructions may be configured to be executed by one or more processors, which when executing the series of computer instructions, performs or facilitates the performance of all or part of the disclosed methods and procedures.

As noted above, the disclosed system in some embodiments relies on one or more modules (hardware, software, firmware, or a combination thereof) to perform various functionalities discussed above.

Those skilled in the art will understand that they can freely combine all aspects of the present invention disclosed herein, without departing from the scope of the invention as disclosed. Further, aspects described for different embodiments of the present invention may be combined. Although the invention has been described by way of example, it should be appreciated that variations and modifications may be made without departing from the scope of the invention as defined in the claims and without diminishing its intended advantages. It is therefore intended that such changes and modifications be covered by the appended claims.

Various preferred features and embodiments of the present invention are described below by way of non-limiting examples.

### EXAMPLES

### Example 1: Converting Food Frequency Intake Data to Nutrients

Food intake data from the participants of a publicly available citizen-science project called the American Gut Project (AGP) (McDonald D, et al. mSystems. 2018) was converted into nutrients intake using a tool called vioscreen and provided to the public by AGP.

### Example 2: Building Models to Estimate Relative Amounts of Faecalibacterium prausnitzii (Fprau)

Predictive models were built to determine the relative amounts of Faecalibacterium prausnitzii (Fprau) of an individual subject. In particular, the model predicted the Fprau relative amounts by several feature parameters to determine whether a subject has “Low” or “notLow”, “High” or “notHigh”; “Low” or “High” Fprau amounts; as per the categories defined above.

A cube-root transformation was performed to make the amounts of Fprau normally distributed before binning them in different categories. The values for various definition of bins were: first/lower quartile −0.2819, third/upper quartile −0.4666, mean-std −0.1954, mean+std −0.5220

For building the classification models, the data was split into a training set “Train” and a testing set “holdout/Test set”. For optimal model performance we used downsampling to balance the imbalanced classes, which may occur based on the definition of bins.

The Train set was used by a machine learning algorithm to train the model. This involved finding variables (i.e., features) and thresholds (or coefficients) to use for classifying the groups. The learning from the data was done in a cross-validated manner where Train data was split into partitions with some parts used for training the model and other for internal testing (k-Fold Cross-Validation, for ex., 3-folds), or with this process also repeated a few times (Repeated k-Fold Cross-Validation, for ex., 10-folds, 10-repeats).

The holdout/Test set was used only for checking the performance of the final trained model. This holdout/Test dataset was thus not used during the model training phase. We evaluated multiple statistical models (different machine learning algorithms) using freely available tools (R software, python) and identified the best models for Low vs. notLow, High vs. notHigh, and Low vs. High for Fprau amounts.

Evaluating the model performance was critical, during all phases of modeling. Once the model was trained, it was applied on holdout/Test data, which was not used during the training phase. The model computed probabilities to be in each group (e.g. “Low”, “notLow”). A final decision was made based on this probability, which thus required the use of a threshold. This threshold impacted the final classification for a subject, whether a subject was correctly classified or not. Thus, the error was evaluated for different choices of threshold. For each given threshold, a confusion matrix was computed. This confusion matrix essentially listed the number of correctly and incorrectly classified subjects. By using different thresholds, one generated many confusion matrices, which were used to derived sensitivity and specificity at different thresholds. These two metrics—sensitivity and specificity—were commonly shown in the form of a Receiving Operating Curve (ROC); which summarized the model performance over several threshold values.

Receiver Operating Characteristic (ROC) curves were produced for the model. We either defined the group of “Low” subjects (and in “notLow” group) and predicted the probability of subject to be in this group; or, we defined the subjects to be in the “High” group (and in “notHigh” group) and predicted the probability of subject to be in this group; or, we defined the subjects to be in the “Low” group (and in “High” group) and predicted the probability of subject to be in this group.

As mentioned earlier, the data set used for the examples of a predictive model was from the American Gut Project (AGP) database (http://americangut.org).

### Example 3: Estimating “Low” Fprau Amounts from Nutrient Intake Data (I)

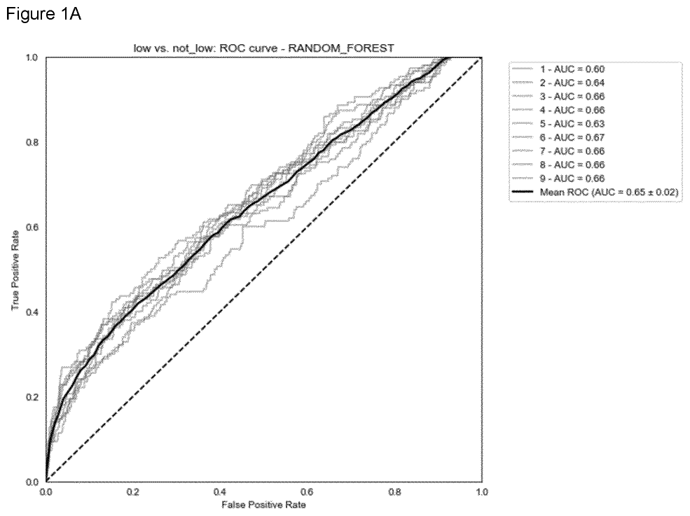

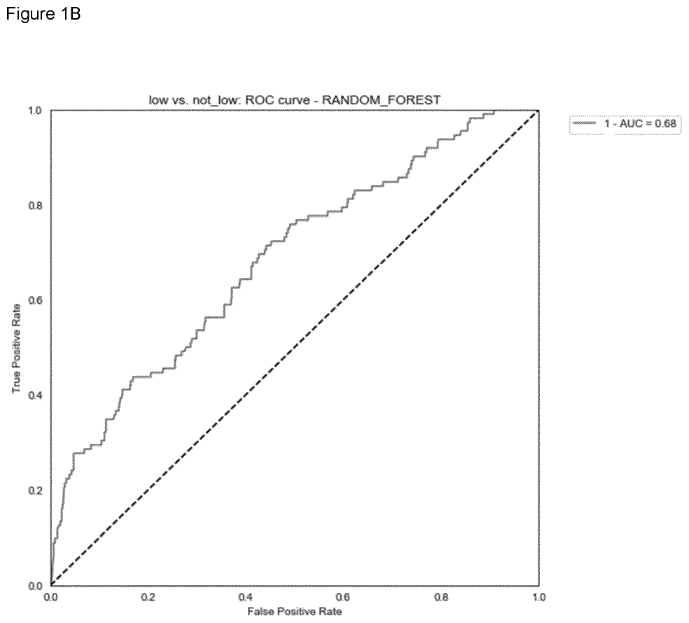

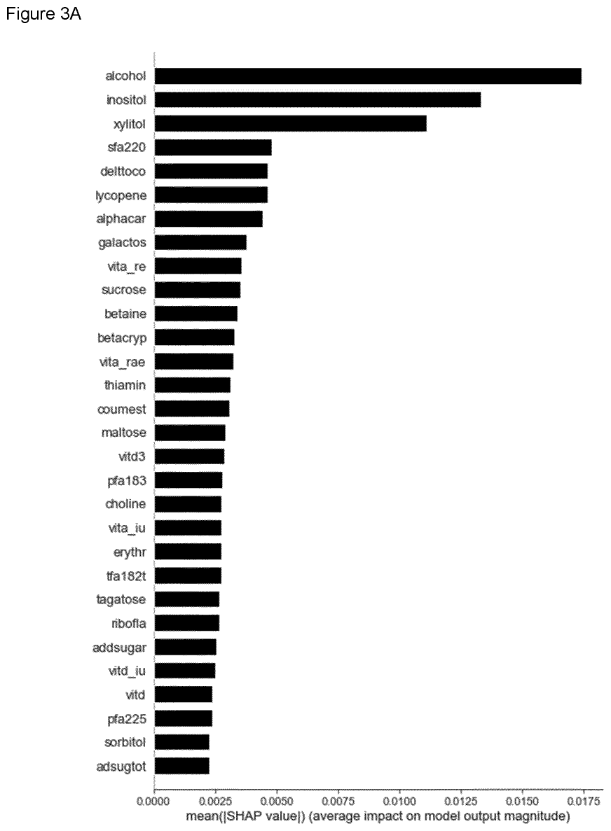

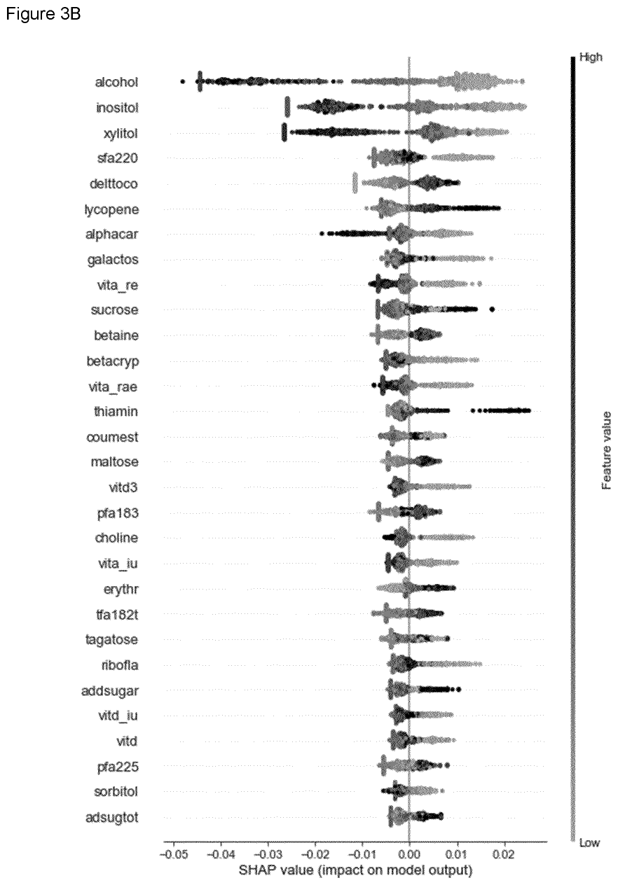

A model for Low vs. notLow Fprau amounts was learned with these parameters: Bin definition: (mean−1*std) vs rest; Features cut-offs: None; Algorithm: RandomForest; Train mode: cv-splits-3, cv-repeats-3; PostProcess Train size: 896, holdout/Test size (original/before preprocess train/test split): 764 (Test percentage: 20.0%). The results obtained for Train in cross-validation were: Accuracy −0.58±0.02, Sensitivity −0.61±0.05, Specificity −0.58±0.03. Train ROC curve is shown in FIG. 1A. The results obtained for holdout/Test set were: Accuracy −0.64, Sensitivity −0.56, Specificity −0.65. holdout/Test ROC curve is shown in FIG. 1B. The important features and their association with Fprau amounts are shown in FIG. 3.

### Example 4: Estimating “Low” Fprau Amounts from Nutrient Intake Data (II)

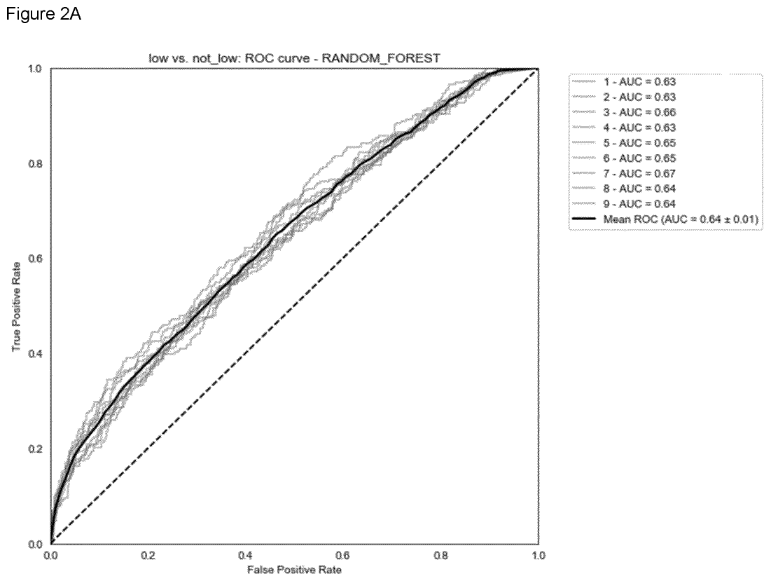

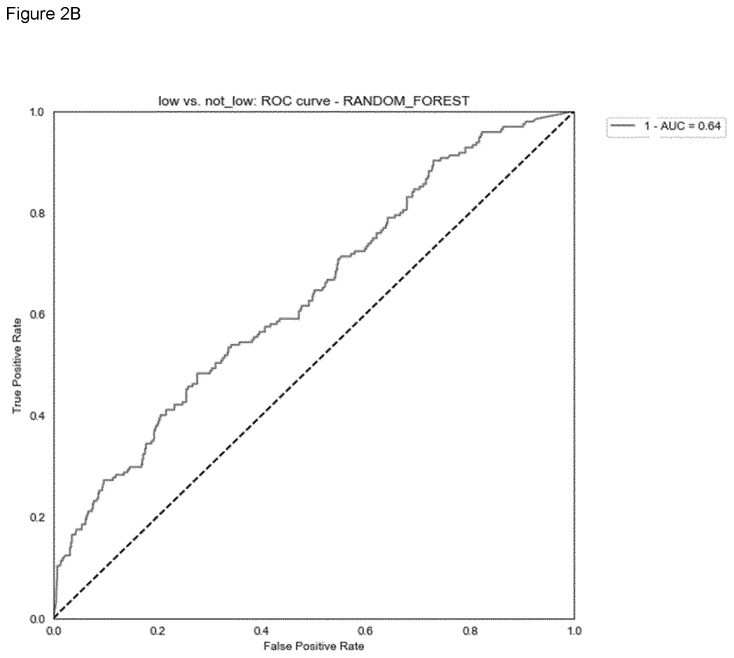

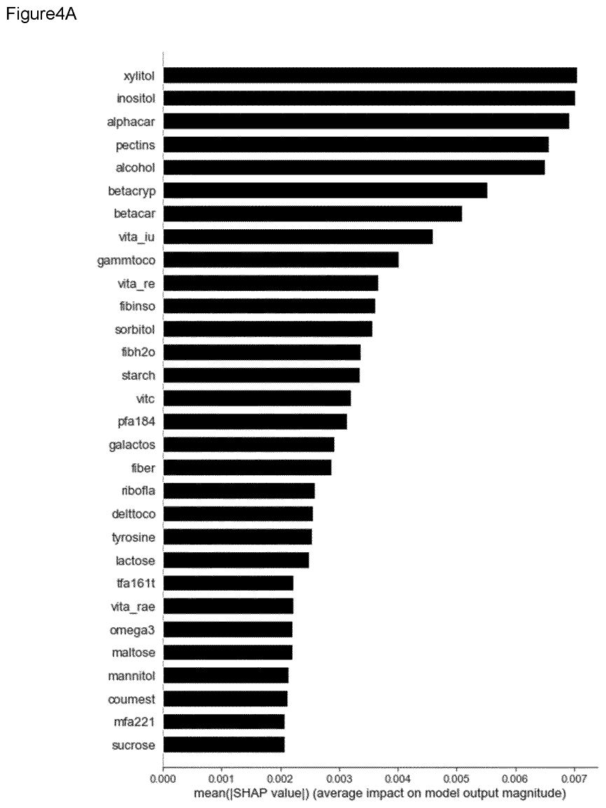

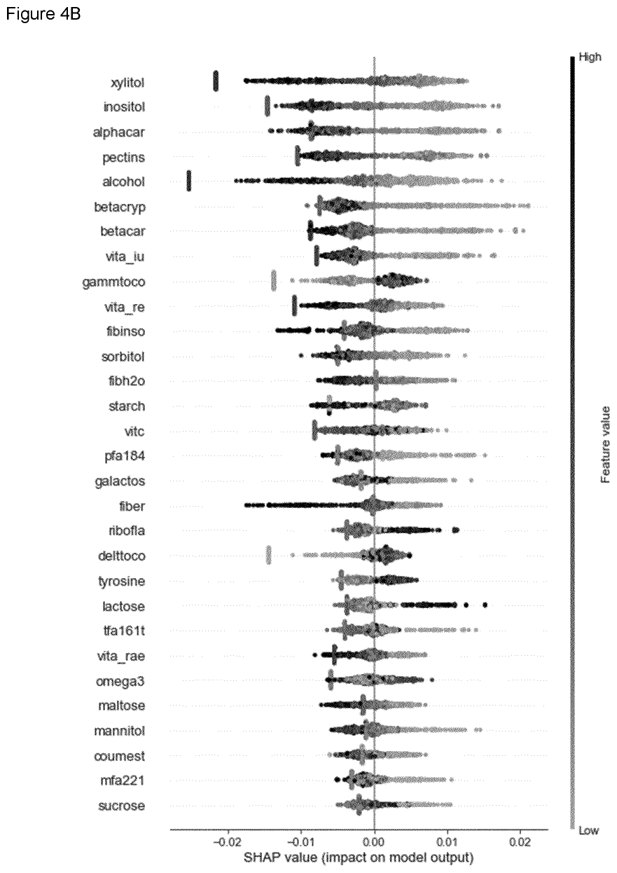

Another model for Low vs. notLow Fprau amounts was learned with these parameters: Bin definition: first/lowest quartile vs rest; Features cut-offs: None; Algorithm: RandomForests; Train mode: cv-splits-3, cv-repeats-3; PostProcess Train size: 1554, holdout/Test size (original/before preprocess train/test split): 764 (Test percentage: 20.0%). The results obtained for Train in cross-validation were: Accuracy −0.58±0.02, Sensitivity −0.62±0.03, Specificity −0.57±0.03. Train ROC curve is shown in FIG. 2A. The results obtained for holdout/Test set were: Accuracy −0.59, Sensitivity −0.57, Specificity −0.59. holdout/Test ROC curve is shown in FIG. 2B. The important features and their association with Fprau amounts are shown in FIG. 4.

### Example 5: Recommendations to Maintain or Improve Fprau Amounts

For the model presented in Example 3, the top 30 features that constitute the model were shown in FIG. 3. For the model presented in Example 4, the top 30 features that constitute the model were shown in FIG. 4. Both (A) and (B) were obtained by running SHapley Additive explanation (SHAP) values analyses (Lundberg S M, et al. Nat Mach Intell. 2020). (A) shows the average impact per feature on the model output in their order of importance from high to low. The main/best feature was the top horizontal bar. The next best feature was the second horizontal bar and so on. (B) shows in more details the impact of a feature per instance/sample on the model output. The color gradation from grey to black indicates low to high values for that feature. The vertical line at 0.00 defines the directionality of impact—to the left is negative impact and to the right is positive impact on the model output. Here, the SHAP analysis output was with respect to the reference class which is “Low”.

If a feature has black values towards the right of the vertical line at 0.00, this indicates higher values of this feature contribute positively to the model output. Vice a versa, if a feature has black values towards the left of the vertical line at 0.00, this indicates higher values of this feature contribute negatively to the model output. Similarly, if a feature has grey values towards the right of the vertical line at 0.00, this indicates lower values of this feature contribute positively to the model output. Vice a versa, if a feature has grey values towards the left of the vertical line at 0.00, this indicates lower values of this feature contribute negatively to the model output.

As can be seen from FIGS. 3 and 4, as an example, some of the important features for this model to predict Low versus notLow Fprau amounts were related to: inositol (Inositol in g), alphacar (Alpha Carotene provitamin A carotenoid in mcg), betacar (Beta Carotene provitamin A carotenoid in mcg), pectins (Pectins in g), fibers (Total Dietary Fiber in g, Soluble Dietary Fiber in g, Insoluble Dietary Fiber in g), and vitamin A (vita_iu—Total Vitamin A Activity in IU, vita_rae—Total Vitamin A Activity Retinol Activity Equivalents in mcg, vita_re—Total Vitamin A Activity Retinal Equivalents in mcg) and so on.

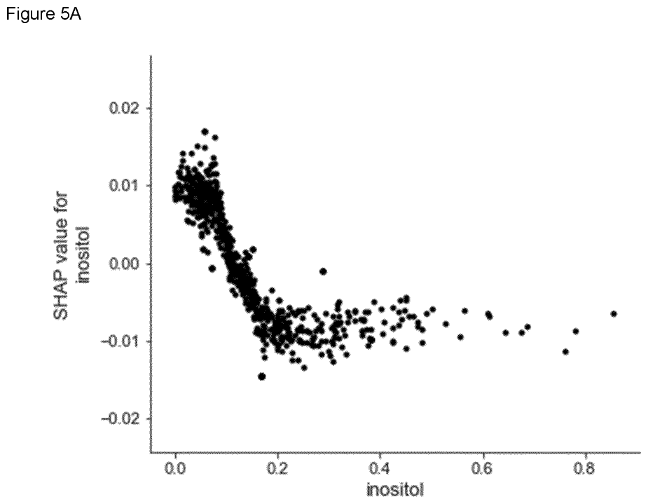

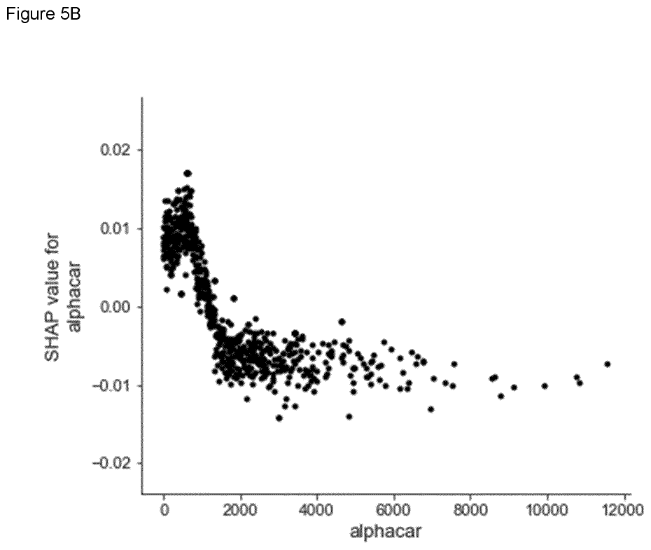

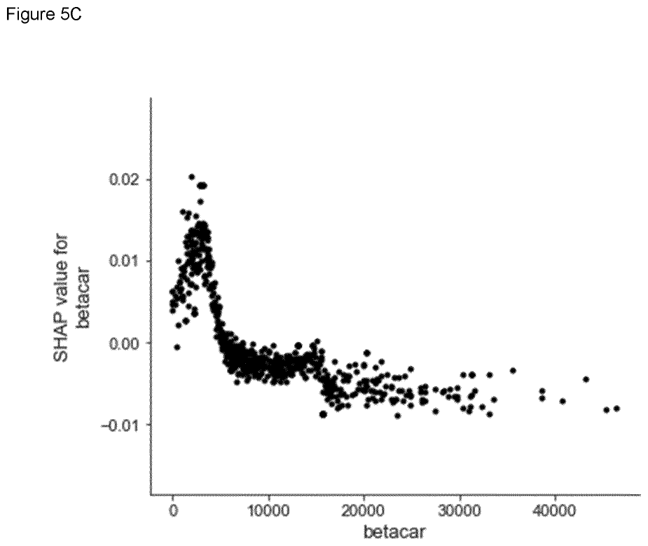

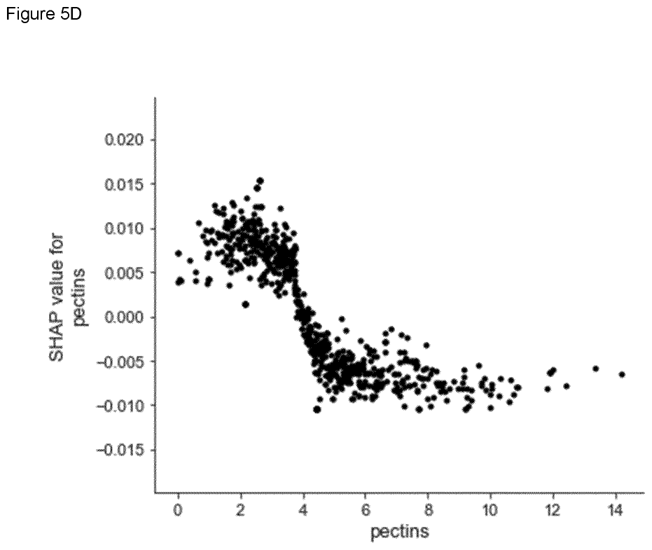

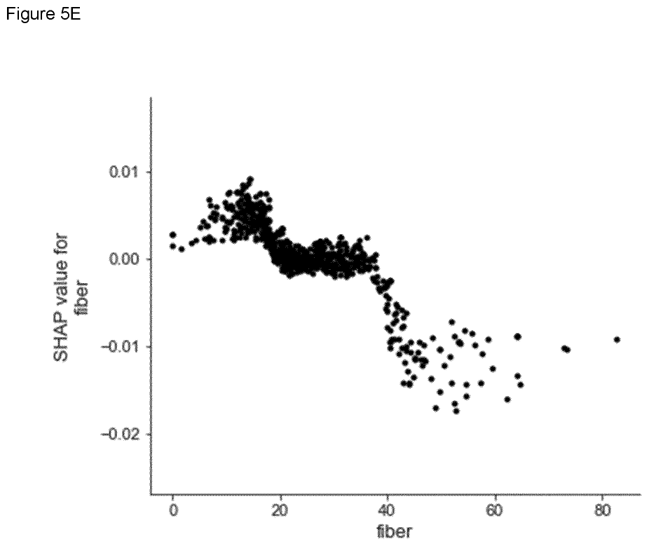

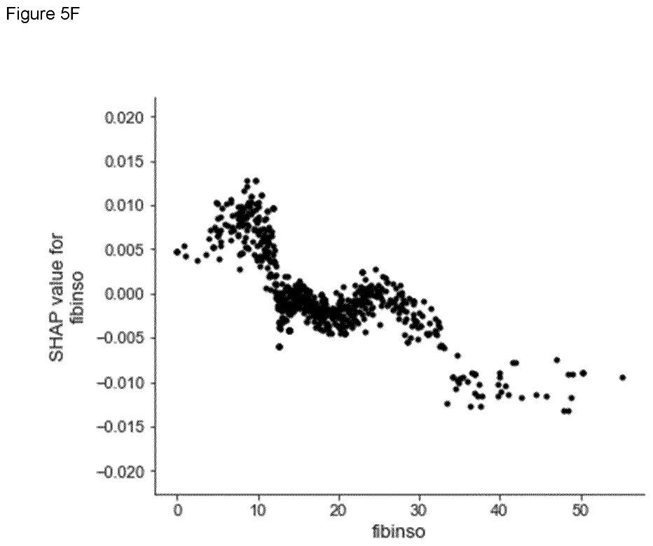

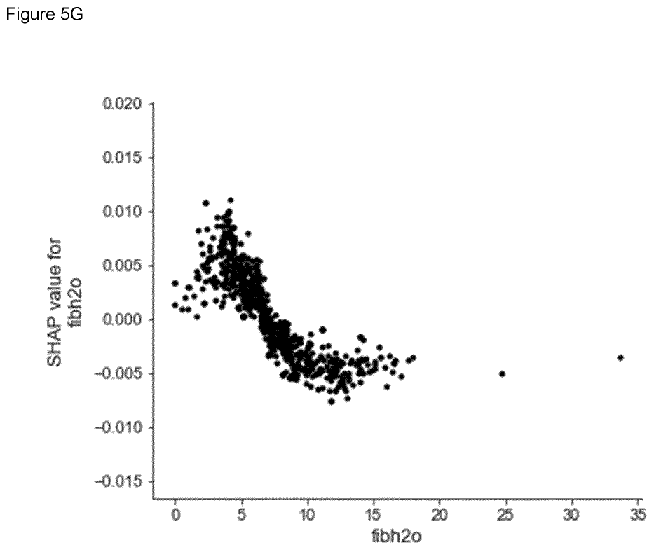

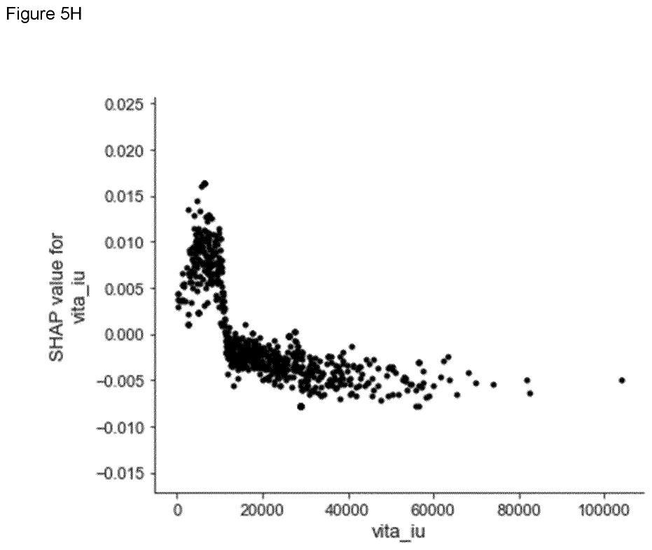

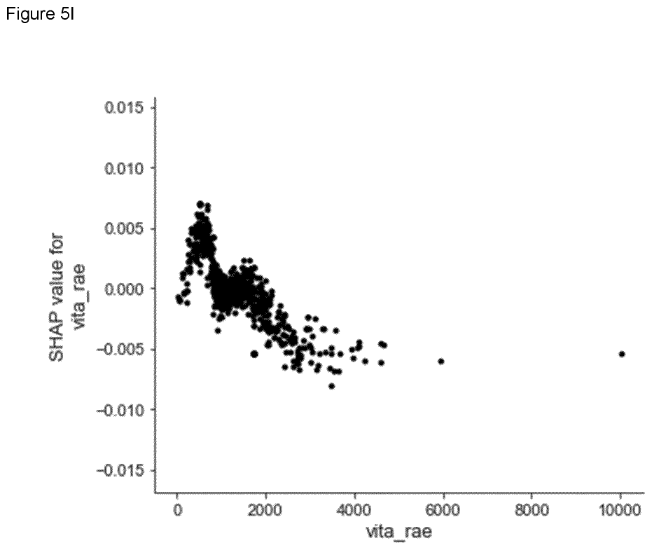

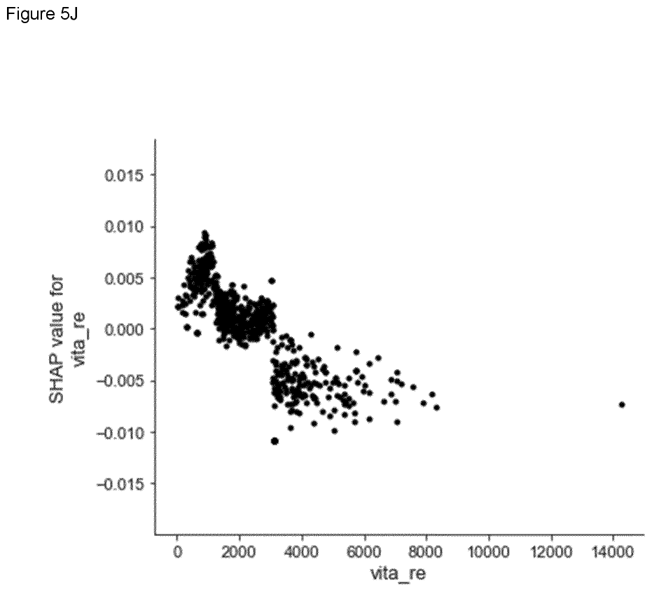

In FIG. 5, per feature, the SHAP Dependence Plot showed for each data instance/sample, the points with the feature value on the x-axis and the corresponding Shapley value on the y-axis. SHAP explained the prediction of each instance by computing the contribution of each feature to the prediction. Shapley value explanation was represented as an additive feature attribution method, as a linear model. The reference class here was “Low”, so the positive coefficients of SHAP value for the corresponding x-values of the feature indicate how much the model was affected by this feature in predicting the “Low” class.

Inositol impacted the Fprau amounts as can be seen here—it was amongst the very top features used by this model (FIGS. 3 and 4). As can be seen in FIG. 5A, specific intake values of inositol had a relation with impact on model output—with low inositol intake tended to have the Fprau status to be on the lower side, while higher intake amounts of inositol tended to have the Fprau status to “notLow” class. Thus, Fprau status would benefit from more inositol intake from diet, preferably more than 0.2 g inositol per day, which can be obtained by eating fruits such as cantaloupe and oranges.

FIGS. 3 and 4 show the importance of alphacar (Alpha Carotene provitamin A carotenoid in mcg) and FIG. 5B depicts the SHAP dependence plot for alphacar (Alpha Carotene provitamin A carotenoid in mcg). For all the individuals with lower intakes of alphacar (all data points on x-axis approximately below the values at 2000), the SHAP values were positive, indicating that this was related to being in the “Low” class of Fprau status. Similarly, only for individuals with higher intakes of alphacar approximately greater than 2000, the SHAP values were negative, indicating that was now related to being in “notLow” Fprau status. Thus, the recommendation of this invention was to consume yellow-orange vegetables such as carrots, sweet potatoes, pumpkin, winter squash and dark-green vegetables such as broccoli, green beans, Green peas, spinach, turnip greens, collards, leaf lettuce and avocado, which are reported to be rich in alpha-carotene.

Based on similar reasoning and explanations above, and looking holistically together at FIGS. 3, 4, 5C, it can be inferred that approximately higher than 10000 mcg consumption of betacar (Beta Carotene provitamin A carotenoid in mcg) was good for the Fprau as it was related to being in the “notLow” microbiome status. Based on these results, the recommendation of the invention was to consume more of yellow and orange fruits, such as cantaloupe, mangoes, pumpkin, and papayas, and orange root vegetables such as carrots and sweet potatoes. Additionally, it is also present in green leaf vegetables such as spinach, kale, sweet potato leaves, and sweet gourd leaves. Further, it is also sold as a dietary supplement. The below table lists the main foods with their amounts of Beta Carotene (https://en.wikipedia.ora/wiki/Beta-Carotene):

| | | | | Milligrams β- |
|---|---|---|---|---|
| | Grams | | Milligrams | carotene per |
| | per | Serving | β- carotene | carotene per |
| Item | serving | size | per serving | serving100 g |
| Carrot juice, canned | 236 | 1 cup | 22.0 | 9.3 |
| Carrots, cooked, boiled, drained, | 156 | 1 cup | 13.0 | 8.3 |
| without salt | | | | |
| Carrots, frozen, cooked, boiled, | 146 | 1 cup | 12.0 | 8.2 |
| drained, without salt | | | | |
| Collards, frozen, chopped, cooked, | 170 | 1 cup | 11.6 | 6.8 |
| boiled, drained, without salt | | | | |
| Pumpkin, canned, without salt | 245 | 1 cup | 17.0 | 6.9 |
| Spinach, canned, drained solids | 214 | 1 cup | 12.6 | 5.9 |
| Spinach, frozen, chopped or leaf, | 190 | 1 cup | 13.8 | 7.2 |
| cooked, boiled, drained, without salt | | | | |
| Sweet potato, canned, vacuum | 255 | 1 cup | 12.2 | 4.8 |
| pack | | | | |
| Sweet potato, cooked, baked in | 146 | 1 potato | 16.8 | 11.5 |
| skin, without salt | | | | |
| Sweet potato, cooked, boiled, | 156 | 1 potato | 14.7 | 9.4 |
| without skin | | | | |

Based on similar reasoning and explanations above, and looking holistically together at FIGS. 4, and 5D, it can be inferred that pectin (Pectins_in_g) consumption was associated with Fprau status. Specifically, increased pectins consumption more than 4 g was associated with “notLow” Fprau status. Thus, the recommendation of the invention would be to consume more pectins for example, from pears, apples, guavas, quince, plums, gooseberries, and oranges and other citrus fruits reported to contain large amounts of pectin. Typical levels of pectin in fresh fruits and vegetables are: Apples-1-1.5%, Apricots—1%, Cherries—0.4%, Oranges—0.5-3.5%, Carrots—1.4%, Citrus peels—30%, Rose hips—15% (https://en.wikipedia.orniwiki/Pectin).

In summary from SHAP analysis shown in FIGS. 3, 4, 5E, F and G, the conclusion was that increasing amounts of fibers positively impacted the microbiome. The fibers in this data were captured as total fibers fiber—Total Dietary Fiber in g (fiber), insoluble fibers—Insoluble Dietary Fiber in g (fibinso), and soluble fibers—Soluble Dietary Fiber in g (fibh20). Based on the interpretations done here, the recommendation of the invention was to have more than 40 g of total fibers, with insoluble fibers being more than 30 g, and soluble fibers being more than 10 g. Thus, the recommendation of the invention would be to take more total fibers composed both of insoluble and soluble fibers to boost the Fprau amounts, which can be obtained from dietary sources. Dietary fibers are found in fruits, vegetables and whole grains. The amount of fiber contained in common foods is listed here (https://en.wikipedia.orgiwiki/Dietary fiber):

| | Serving | | Fibermass |
|---|---|---|---|
| Food group | mean | | per serving |
| Fruit | 120 mL | (0.5 cup) | 1.1 g |
| Dark green vegetables | 120 mL | (0.5 cup) | 6.4 g |
| Orange vegetables | 120 mL | (0.5 cup) | 2.1 g |
| Cooked dry beans (legumes) | 120 mL | (0.5 cup) | 8.0 g |
| Starchy vegetables | 120 mL | (0.5 cup) | 1.7 g |
| Other vegetables | 120 mL | (0.5 cup) | 1.1 g |
| Whole grains | 28 g | (1 oz) | 2.4 g |
| Meat | 28 g | (1 oz) | 0.1 g |

Soluble fiber is found in varying quantities in all plant foods, including legumes (peas, soybeans, lupins and other beans), oats, rye, chia, and barley, some fruits (including figs, avocados, plums, prunes, berries, ripe bananas, and the skin of apples, quinces and pears), certain vegetables such as broccoli, carrots, and Jerusalem artichokes, root tubers and root vegetables such as sweet potatoes and onions (skins of these are sources of insoluble fiber also), psyllium seed husks (a mucilage soluble fiber) and flax seeds, nuts, with almonds being the highest in dietary fiber.

Sources of insoluble fiber include: whole grain foods, wheat and corn bran, legumes such as beans and peas, nuts and seeds, potato skins, lignans, vegetables such as green beans, cauliflower, zucchini (courgette), celery, and nopal, some fruits including avocado, and unripe bananas, the skins of some fruits, including kiwifruit, grapes and tomatos.

Similarly, FIGS. 5H, I, J indicate increasing amounts of Vitamin A have a desired effect on Fprau amounts. This was captured in AGP data as vita_iu (Total Vitamin A Activity in IU), vita_rae (Total Vitamin A Activity Retinol Activity Equivalents in mcg), and vita_re (Total Vitamin A Activity Retinol Equivalents in mcg). 1 IU of retinol is equivalent to approximately 0.3 micrograms (300 nanograms). As per FIGS. 5H, I, J vita_iu>20000 IU, vita_rae>2000 mcg, and vita_re>3000 mcg had desired effects on Fprau amounts.

Dietary vitamin A is sourced from two sources. Animal products have the active forms, as retinoids and include retinaldehyde and retinol, are quickly available. Precursors must be converted to active forms, called as provitamins, are obtained from fruits and vegetables containing yellow, orange and dark green pigments, known as carotenoids, the most well-known being p-carotene. Amounts of vitamin A are measured in Retinol Equivalents (RE).

One RE is equivalent to 0.001 mg of retinol, or 0.006 mg of (β-carotene, or 3.3 International Units of vitamin A. Retinoids are found naturally only in foods of animal origin. Each of the following contains at least 0.15 mg of retinoids per 1.75-7 oz (50-198 g): Cod liver oil, Butter, Liver (beef, pork, chicken, turkey, fish), Eggs, Cheese, and Milk.

Thus, the recommendation of the invention was to eat animal products, such as eggs, liver, cod liver oil. Additionally, synthetic retinol is available commercially as: Acon, Afaxin, Agiolan, Alphalin, Anatola, Aoral, Apexol, Apostavit, Atav, Avibon, Avita, Avitol, Axerol, Dohyfral A, Epiteliol, Nio-A-Let, Prepalin, Testavol, Vaflol, Vi-Alpha, Vitpex, Vogan, and Vogan-Neu. (https://en.wikiDedia.ora/wiki/Retinol)

The final recommendation is the result of a complex multivariate analysis where features were related to each other and the final impact on the Faecalibacterium prausnitzii (Fprau) status of an individual was a combination of different factors.

The system of the present invention with its user-friendly digital interface would incorporate these recommendations to communicate them directly with the user for improving their microbiome status.

### Example 6

Stool samples were collected from a healthy adult donor under human study protocol. Upon the reception of the stool samples, small aliquots were prepared with storage buffer (PBS and 10% glycerol) and were stored at −80 degrees Celsius before use. For each experiment, 250 μL of stool aliquot was inoculated in a 10 mL Hungate tube filled with a minimal bacteria culture media under a strict anaerobic condition (Oxygen <3 ppm) inside of an anaerobic chamber. Different nutrients or nutrient combinations, shown in Table 1, were added to the culture media at time 0 and the tubes were incubated at 37 degrees Celsius for 24 or 48 hours. The growth of Faecalibacterium prausnitzii (F. prau) was examined by two methods: quantitative PCR specifically targeting F. prau and 16S microbial rRNA gene sequencing.

TABLE 1
nutrients or nutrient combinations tested
in an in vitro fermentation experiment

| Nutrient group | Ingredient |
|---|---|
| Control | Only minimal media without addition of |
| | nutrients (negative control) |
| PuMP_Full | Minimal media plus inositol, vit B5, B6, |
| | B12, vit A and vit E |
| Vit Bs+ inositol | Minimal media plus vit B5, B6, B12 and inositol |
| Vit Bs | Minimal media plus vit B5, B6 and B12 |
| Inulin | Minimal media plus inulin (positive control) |
| inositol | Minimal media plus inositol |

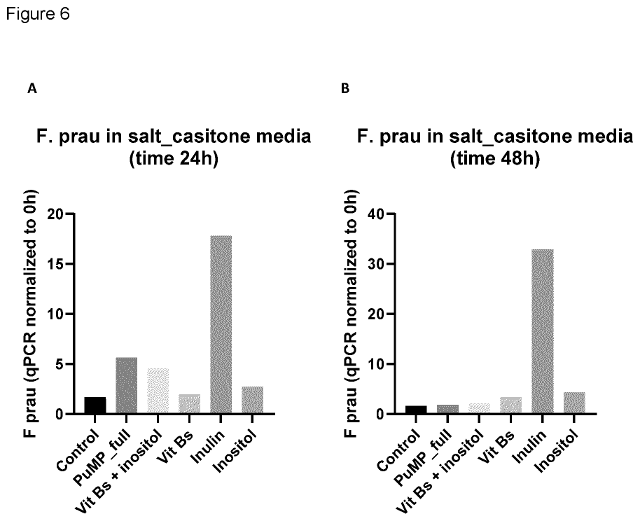

First, we examined the absolute quantity of F. prau in the community after 24 or 48 hours (FIGS. 6A and 6B). At 24 hours, F. prau in PuMP_full, Vit Bs+inositol and inulin are at least twice as much as control. However, only inulin was able to maintain a high amount of F. prau after 48 hours.

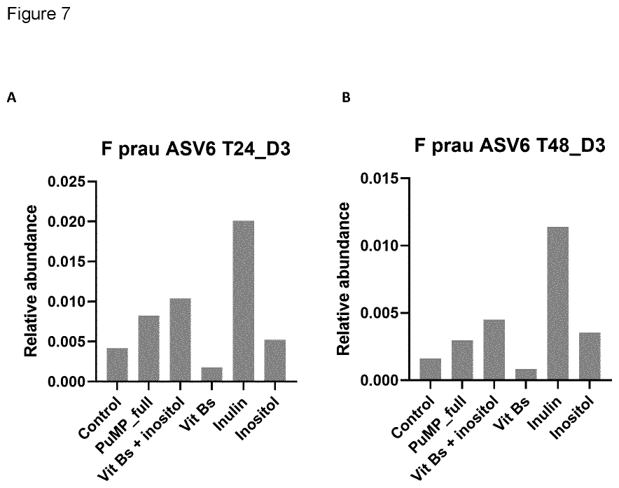

F. prau is heterogenic and genetically diverse. Thus, in this next experiment, we examined if nutrient or nutrient combinations favor specific F. prau to grow in a mixed community. A total of 14 genetically different Faecalibacterium were found in the fermentation experiment and most of them (ASV1, 6, 9, 12 and 13) responded positively to inulin as expected. Interestingly, ASV6 also responded to PuMP_full and vit Bs+inositol at 24 hours (FIG. 7A) and to a lesser extent at 48 hours (FIG. 7B).

In conclusion, our results demonstrates that specific nutrient combinations (PUMP_full and vit Bs+inositol) provides an advantage for F. prau to grow in a mixed community although the effect did not sustain to 48 hours. More importantly, the benefits of these nutrient combinations are visible only in certain F. prau but not all F. prau, suggesting that these nutrient combinations can be used in combination with F. prau boosting fiber such as inulin or by themselves independently when inulin cannot be used in a product or is not tolerated by people.

1. A method for determining the gut Faecalibacterium prausnitzii (Fprau) status comprising:
(i) determining the gut Fprau status in a subject and
(ii) providing recommendations to improve or maintain Fprau status in said subject.

2. Method according to claim 1 wherein the determination of gut Fprau status is by a food frequency questionnaire to determine the nutrients intake to predict the Fprau status of said subject.

3. Method according to claim 1 wherein the determination of gut Fprau status is additionally by a biological sample to quantify the microbiome diversity of said subject.

4. Method according to claim 1 wherein said method is computer-implemented.

5. Method according to claim 1 wherein said method involves evaluation of feature parameters related to gut Fprau status as low, medium or high.

6. A computer implemented method comprising:
(i) determination of the gut Fprau status in a subject;
(ii) providing a recommendation of a personalized fiber composition; and
(iii) delivering a personalized nutritional recommendation.

7. (canceled)

8. A method for optimizing one or more dietary interventions for a subject comprising:
(i) determining the Fprau status of a subject according to a method comprising:
determining the gut Fprau status in a subject and
providing recommendations to improve or maintain Fprau status in said subject; and
(ii) applying the dietary intervention to the subject.

9. A method according to claim 1 wherein the recommendation is a nutritional composition selected from the group consisting of: food products, beverage products, or dietary supplements or a combination thereof in a kit of parts delivered to the individual.

10. Method according to claim 1 wherein said dietary intervention comprises recommendations for food, nutrient groups, recipe or meal plans selected from the group consisting of:
(i) consuming total fibers composed both of insoluble and soluble fibers;
(ii) following Mediterranean diet;
(iii) consuming food containing pectins;
(iv) drinking red wine;
(v) consuming raisins;
(vi) eat animal products;
(vii) increasing amounts of Vitamin A;
(viii) increasing consumption of alpha-carotene; and
(ix) increasing consumption of beta-carotene.
